(19) United States
(12) Patent Application Publication
(10) Pub. No.: US 2021/0374955 A1
(43) Pub. Date: Dec. 2, 2021

(54) RETINAL COLOR FUNDUS IMAGE ANALYSIS FOR DETECTION OF AGE-RELATED MACULAR DEGENERATION

(71) Applicant: Zasti Inc., Oakton, VA (US)

(72) Inventors: Ramanathan Krishnan, Oakton, VA (US); John Domenech, Big Pine Key, FL (US); Rajagopal Jagannathan, Tamil Nadu (IN)

(21) Appl. No.: 17/337,237

(22) Filed: Jun. 2, 2021

Related U.S. Application Data
(60) Provisional application No. 63/033,447, filed on Jun. 2, 2020.

(51) Int. Cl. G06T 7/00; G16H 50/30

(52) U.S. Cl. CPC G06T 7/0012; G16H 50/30; G06T 2207/30041; G06T 2207/10101; G06T 2207/20084; G06T 2207/30096; G06T 2207/20081

(57) ABSTRACT

A facility diagnoses AMD in a subject patient. The facility obtains one or more patient images for a subject patient, which depict at least one of the subject patient's eyes. The facility applies an image-based classifier to at least one of the patient images to obtain a first AMD risk score. The facility identifies the macular region of an eye depicted in the patient images, and applies a deep learning-based classifier to the identified macular region to obtain a second AMD risk score. The facility identifies lesions present in an eye depicted in the patient images, and applies a deep learning-based classifier to the identified lesions to obtain a third AMD risk score. The facility combines the first AMD risk score, second AMD risk score, and third AMD risk score to obtain a unified AMD risk score.

### CROSS-REFERENCE TO RELATED APPLICATIONS

This Application claims the benefit of U.S. Provisional Application 63/033,447, filed Jun. 2, 2020 and entitled “RETINAL COLOR FUNDUS IMAGE ANALYSIS FOR DETECTION OF AGE-RELATED MACULAR DEGENERATION,” which is hereby incorporated by reference in its entirety.

In cases where the present application conflicts with a document incorporated by reference, the present application controls.

### BACKGROUND

With advancements in the medical field and the increase in life expectancy, age-related diseases also tend to become more common. Age-related macular degeneration (AMD) is one of the major causes for blindness in the elderly population. Early detection is very important for prevention and treatment of AMD.

Conventional approaches to monitoring retinal diseases and detecting AMD use a variety of different imaging techniques, such as Color Fundus Imaging (CFI) and Optical Coherence Tomography (OCT), to monitor retinal diseases; retinal specialists manually inspect the retina to look for signs of the disease.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram showing some of the components typically incorporated in at least some of the computer systems and other devices on which the facility operates.

FIG. 2 is a block diagram depicting an example AMD ensemble model trained and applied by the facility in some embodiments.

FIG. 3 is a flow diagram showing a process performed by the facility in some embodiments to create a fundus classification module used to generate a first AMD risk score.

FIG. 4 is a block diagram depicting an example fundus image classification module trained and applied by the facility in some embodiments.

FIG. 5 is a flow diagram showing a process performed by the facility in some embodiments to create a macular extraction module used to generate a second AMD risk score.

FIG. 6 is a display diagram showing a sample distance map used by the facility to locate the fovea in some embodiments.

FIG. 7 is a display diagram depicting a GAN framework used to locate the fovea, used by the facility in some embodiments.

FIG. 8 is a flow diagram showing a process performed by the facility in some embodiments to create a lesion extraction module used to generate a third AMD risk score.

FIG. 9 is a lesion segmentation GAN framework used by the facility in some embodiments.

FIG. 10 is a flow diagram showing a process performed by the facility in some embodiments to use an ensemble model to obtain an AMD risk score for a subject patient.

### DETAILED DESCRIPTION

As life expectancy has increased, and age-related diseases have become more common, detecting and treating age-related diseases has imposed an additional burden on healthcare providers. Early detection and treatment of age-related diseases, such as AMD, assist in easing this burden on healthcare providers. With regards to AMD specifically, early detection is important both for prevention of AMD and treatment of the disease. The inventors have recognized a variety of disadvantages to current methods of diagnosing retinal diseases including AMD. First, it is difficult for retinal specialists to diagnose AMD based on only one imaging technique. As a result, retinal specialists often use more than one imaging technique, such as by using both CFI and OCT, in order to diagnose AMD. Additionally, the task of detecting abnormalities in the retina, such as drusen, exudate, hemorrhage, etc., is a labor-intensive and time-consuming process. Furthermore, other methods of diagnosing AMD rely on the Age-Related Eye Disease Study Simplified Severity Scale to predict the risk of progression to late AMD, but do not detect abnormalities occurring in the retina due to AMD. These methods also do not analyze the macula region around the fovea, where the disease tends to predominately occur.

In response to recognizing these disadvantages, the inventors have conceived and reduced to practice a software and/or hardware facility for computer aided diagnosis (CAD) of AMD using retinal color fundus images (“the facility”). The facility enables a retinal specialist to quickly diagnose AMD by generating a score representing a patient's risk of AMD. In some embodiments, the facility obtains the risk score by analyzing the entire retinal fundus image obtained for a patient.

In some embodiments, to obtain the risk score, the facility employs deep learning-based techniques. In some embodiments, the facility includes three parallel modules, a fundus image classification module, a macula extraction module, and a lesion extraction module.

In some embodiments, in the fundus image classification module, the facility employs the whole retinal fundus image and builds an image-based classifier to predict the risk scores for AMD. In some embodiments, the facility augments the image dataset used in the fundus image classification module by performing one or more of: 1) random flipping and rotation, 2) photometric distortion, and 3) specific histogram based processing techniques, such as histogram equalization, adaptive histogram equalization, intensity rescaling at different levels, histogram matching etc. In some embodiments, the facility employs pre-trained deep convolutional neural networks for binary classification such as: 1) EfficientNets, 2) Inception-Resnet, 3) Resnext, and 4) Squeeze and Excitation networks. In some embodiments, the facility combines the predictions of each pre-trained network to obtain a prediction of a risk score. In some embodiments, the predictions are combined by using averaging of posterior probabilities.

In some embodiments, since AMD abnormalities predominantly occur in the macular region, the facility uses a macula extraction module to extract the macular region and then uses the extracted region to predict a risk score for AMD. In some embodiments, for the macula extraction module, the facility utilizes a novel generative adversarial network (GAN) based framework to extract the macular region.

In some embodiments, when extracting the macular region, the facility locates the fovea, such as by predicting the point coordinates of the location of the fovea. In some embodiments, the facility locates the fovea through standard coordinate regression. In some embodiments, the faculty utilizes image-to-image translation to locate the fovea. In some embodiments, the facility creates one or more distance maps having the same size as the fundus images using a Euclidean distance transform computed from the fovea location. In some embodiments, the facility truncates the distance map such that it only contains a specific radius around the fovea.

In some embodiments, the facility then utilizes paired image-to-image translation to locate the fovea. In some embodiments, the facility use a GAN framework to perform image translation. In some embodiments, the facility crops the images around the fovea and passes them to a deep learning-based classifier to obtain risk scores for AMD.

In some embodiments, in a lesion extraction module, the facility extracts lesions such as drusen, scar, exudates etc., and then produces a risk score for AMD based on the properties of extracted lesions. In some embodiments, the facility performs the task of lesion extraction by utilizing fully convolutional networks for semantic segmentation of different lesions. In some embodiments, the facility segments various types of lesions from a fundus image. In some embodiments, the facility utilizes GAN-based frameworks for lesion segmentation. In some embodiments, the facility utilizes strided deconvolutional layers for upsampling. In some embodiments, the facility utilizes one or more of batch normalization, Relu operations, and tanh activation in the GAN-based framework. In some embodiments, the facility trains a GAN for each lesion segmentation task separately. In some embodiments, the facility discards segmentation predictions where the lesion area is less than a specific threshold value found empirically. In some embodiments, the facility semantically segments out the retinal lesions. In some embodiments, the facility presents the segmented retinal lesions to a user.

In some embodiments, the facility builds a lesions-based classifier by passing the segmentation maps to a deep learning-based classifier which assigns a risk score for AMD based only on the lesion segmentation maps.

In some embodiments, the facility combines the risk scores obtained from various modules, such as a macula extraction module, fundus image classification module, and a CNN based lesion extraction module, to produce a unified AMD risk score. In some embodiments, the facility produces the unified

AMD risk score by determining the weighted average of the AMD risk scores obtained from the three deep learning-based classifiers. In some embodiments, the facility utilizes OCT in addition to, or instead of, color fundus imaging to detect AMD.

By performing in some or all of the ways described above, the facility allows retinal specialist to quickly obtain a score representing the probability that a subject patient has AMD.

Also, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by automatically determining a risk score for a subject patient, the facility is able to reduce the amount of computing equipment used by retinal specialists to manipulate and analyze OCT and color fundus images to manually diagnose AMD or determine a patients risk for AMD.

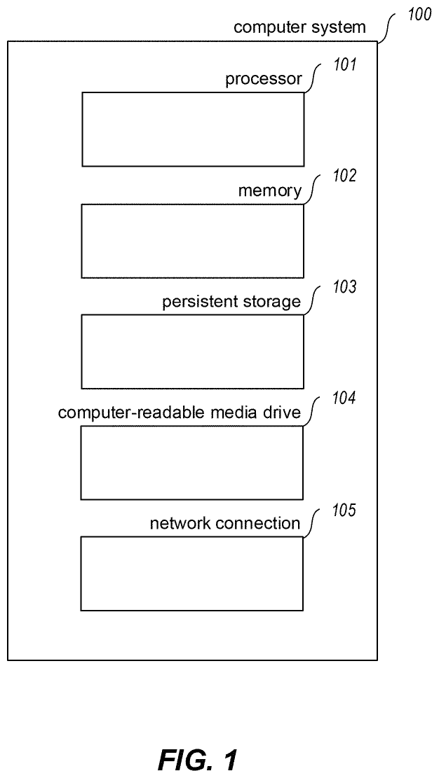

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102 for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

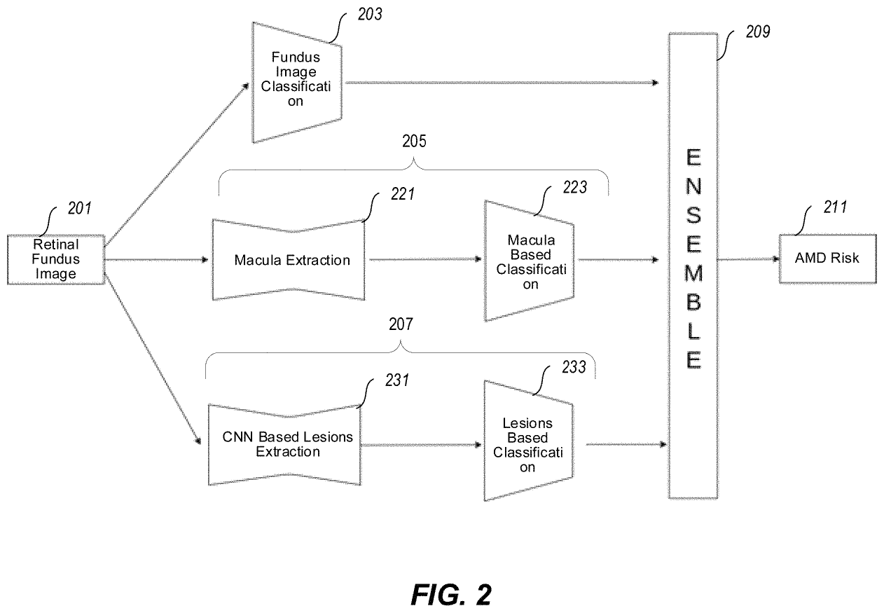

FIG. 2 is a block diagram depicting an example AMD ensemble model trained and applied by the facility in some embodiments. The AMD ensemble model receives one or more images 201; includes a fundus image classification module 203, a macula extraction module 205, a lesion extraction module 207, and an ensembling module 209; and produces a unified AMD risk score 211. The macula extraction module 205 includes a macula extraction block 221 and a macula classification block 223. The lesion extraction module 207 includes a lesion extraction block 231 and a lesion classification block 233.

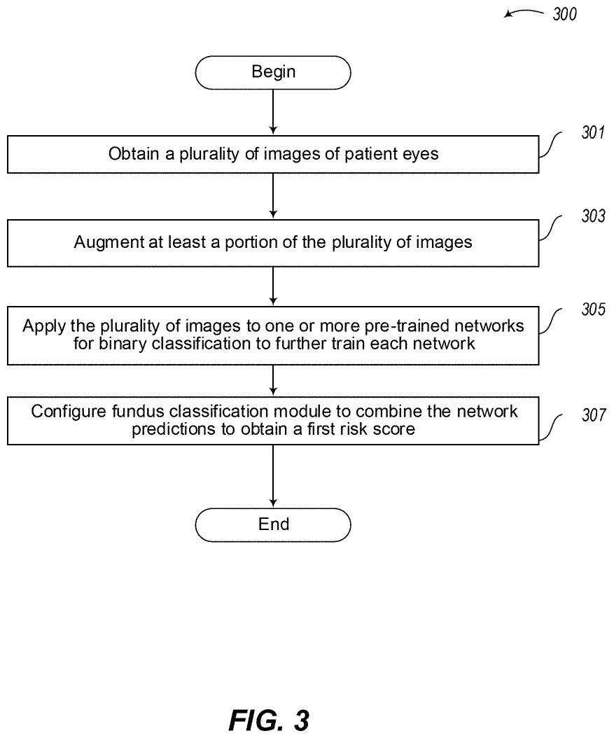

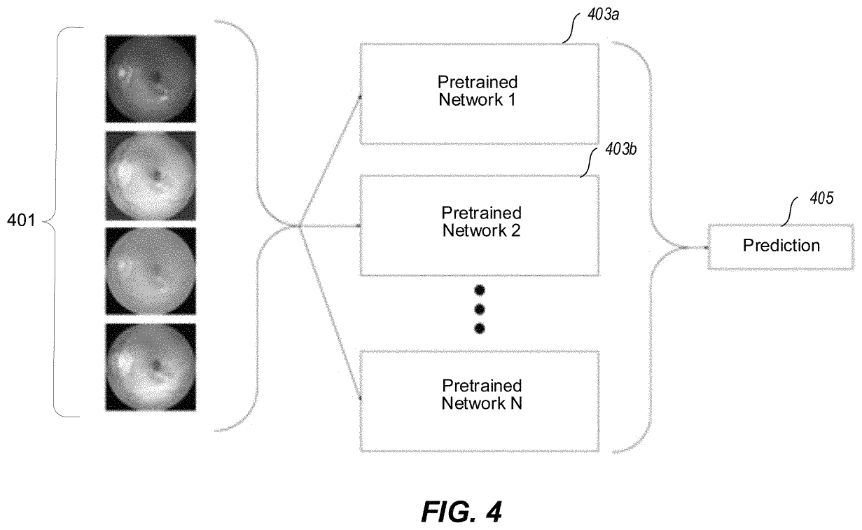

The images 201 are images depicting at least one eye of a subject patient, such as retinal fundus images, images obtained via color fundus imaging, images obtained via OCT, or images obtained via other imaging techniques for obtaining an image of a patient's eye. The fundus image classification module 203 analyzes at least one of the images 201 and generates a first AMD risk score for the subject patient. The fundus image classification module 203 is discussed in more detail in FIGS. 3 and 4.

The macula extraction module 205 identifies the macular region of the subject patient's eyes in at least one of the images 201 in the macula extraction block 221. In the macula classification block 223, the macula extraction module generates a second AMD risk score based on the identified macular region.

In the lesion extraction block 231, the lesion extraction module 231 identifies lesions in the subject patient's eyes, such as a drusen, scar, exudates etc. In the lesion classification block 233, the lesion extraction module generates a third AMD risk score for the subject patient. In the ensembling module 209, the first AMD risk score, second AMD risk score, and third AMD risk score are combined to create the unified AMD risk score 211. In some embodiments, the ensembling module 209 combines the AMD risk scores by obtaining an average of the AMD risk scores, such as a simple average, a weighted average, or other methods of combining risk scores or probabilities. The facility uses the unified AMD risk score to predict whether the subject patient is likely to develop AMD. Furthermore, the prediction of whether the subject patient is likely to develop AMD may be used by medical personnel to suggest or alter treatment options, prevention options, or other medical advice related to AMD. FIG. 3 is a flow diagram showing a process performed by the facility in some embodiments to create a fundus classification module used to generate a first AMD risk score. At act 301, the facility obtains one or more images of patient eyes. In some embodiments, the facility obtains the images by using color fundus imaging, OCT, or other imaging techniques for obtaining images of eyes.

At act 303, the facility augments at least a portion of the obtained images. In some embodiments, the facility augments the images by performing one or more of: random flipping, random rotation, photometric distortion, or other image augmentation techniques. In some embodiments, the facility augments the images by using one or more specific histogram based image processing techniques, such as: histogram equalization, adaptive histogram equalization, intensity rescaling at different levels, histogram matching, and other histogram based image processing techniques.

At act 305, the facility applies the augmented images to one or more pre-trained networks for binary classification to further train each network to detect AMD based on the augmented images. In some embodiments, the networks are pretrained deep convolutional neural networks (CNN), such as ImageNet networks. In some embodiments, the prediction is a prediction of whether AMD is present in the subject patient's eye. In some embodiments, the networks include one or more of: EfficientNets, such as those described in Tan, M., et al.; Inception-Resnet, such as those described in Szegedy, C., et al.; Resnext, such as the architecture described in Xie, S., et al.; Squeeze and Excitation networks, such as those described in Hu, J., et al.; or other classification networks or CNNs.

An EfficientNet is a class of networks which employ a model scaling method to scale up CNNs. In some embodiments, the facility uses multiple classes of EfficientNets, such as EfficientNet-B4, EfficientNet-B5, EfficientNet-B6, EfficientNet-B7. Tan, M., Le, Q.V.: Efficientnet: Rethinking model scaling for convolutional neural networks. arXiv preprint arXiv:1905.11946 (2019), herein incorporated by reference in its entirety.

An Inception-Resnet is an architecture which combines an inception block and a residual block to help perform the classification. The inception block improves multiscale feature extraction, while the residual block improves in convergence and alleviating vanishing gradients. In some embodiments, the inception block and residual block improve the feature extraction process performed by the fundus image classification module. Szegedy, C., loffe, S., Vanhoucke, V., Alemi, A.: Inception-v4, inception-resnet and the impact of residual connections on learning (2016). arXiv preprintarXiv:1602.07261 (2016), herein incorporated by reference in its entirety.

The Resnext architecture is a modularized network architecture for image classification. In some embodiments, the facility uses a pretrained Resnext network which uses pretrained weights obtained by weakly supervised learning to perform the binary classification. Xie, S., Girshick, R., Dollár, P., Tu, Z., He, K.:

Aggregated residual transformations for deep neural networks. In: Proceedings of the IEEE conference on computer vision and pattern recognition. pp. 1492-1500 (2017), herein incorporated by reference in its entirety. Mahajan, D., Girshick, R., Ramanathan, V., He, K., Paluri, M., Li, Y., Bharambe,A., van der Maaten, L.: Exploring the limits of weakly supervised pretraining. In:Proceedings of the European Conference on Computer Vision (ECCV). pp. 181-196(2018), herein incorporated by reference in its entirety.

Squeeze and Excitation networks use squeeze-and-excitation blocks which generalize well across different datasets. These blocks improve pattern recognition by adaptively adjusting the weights for each feature map. Hu, J., Shen, L., Sun, G.: Squeeze-and-excitation networks. In: Proceedings of the IEEE conference on computer vision and pattern recognition. pp. 7132-7141 (2018), herein incorporated by reference in its entirety.

At act 307, the facility configures the fundus classification module to combine the network predictions to obtain a first AMD risk score. In some embodiments, the network predictions are combined by using simple averaging of posterior probabilities. After act 307, the process concludes.

Those skilled in the art will appreciate that the acts shown in FIG. 3 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

FIG. 4 is a block diagram depicting an example fundus image classification module trained and applied by the facility in some embodiments. The fundus image classification module receives one or more images 401, includes one or more pretrained networks 403a and 403b, and produces a prediction 405. The facility applies the pretrained networks 403a and 403b to at least one of the images 401. In some embodiments, the facility augments images 401 before being applying the pretrained networks 403a and 403b to them, such as by performing one or more of: 1) random flipping and rotation, 2) photometric distortion, and 3) specific histogram based processing techniques, such as histogram equalization, adaptive histogram equalization, intensity rescaling at different levels, histogram matching etc. In some embodiments, the facility applies the pretrained networks to altered and unaltered images.

The pretrained networks are machine learning models, neural networks, artificial intelligence, etc., which obtain one or more input images and output a prediction for AMD risk. In some embodiments, the facility obtains the network with pretrained weights, such as ImageNet pretrained weights. In some embodiments, the facility trains the networks to predict whether a subject patient has AMD based on images of other subject patients. In some embodiments, the facility augments or alters the images used to train the networks in a similar manner as act 303. In some embodiments, the pretrained networks include binary classification such as EfficientNets, Inception-Resnet, Resnext, Squeeze and

Excitation networks, or other classification networks. The fundus image classification module combines each of the predictions into one prediction 405. In some embodiments, the facility combines the network predictions by using simple averaging of posterior probabilities.

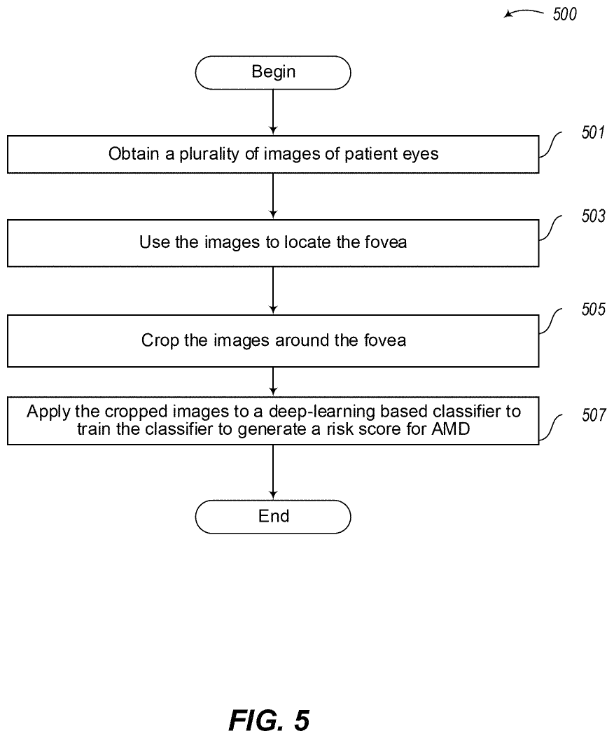

FIG. 5 is a flow diagram showing a process performed by the facility in some embodiments to create a macular extraction module used to generate a second AMD risk score. At act 501, the facility obtains a plurality of images of patient eyes. The facility performs act 501 in a similar manner to act 301.

At act 503, the facility uses the images to locate the fovea. In some embodiments, to locate the fovea, the facility predicts the point coordinates of the location of the fovea. In some embodiments, the facility uses a distance map to locate the fovea.

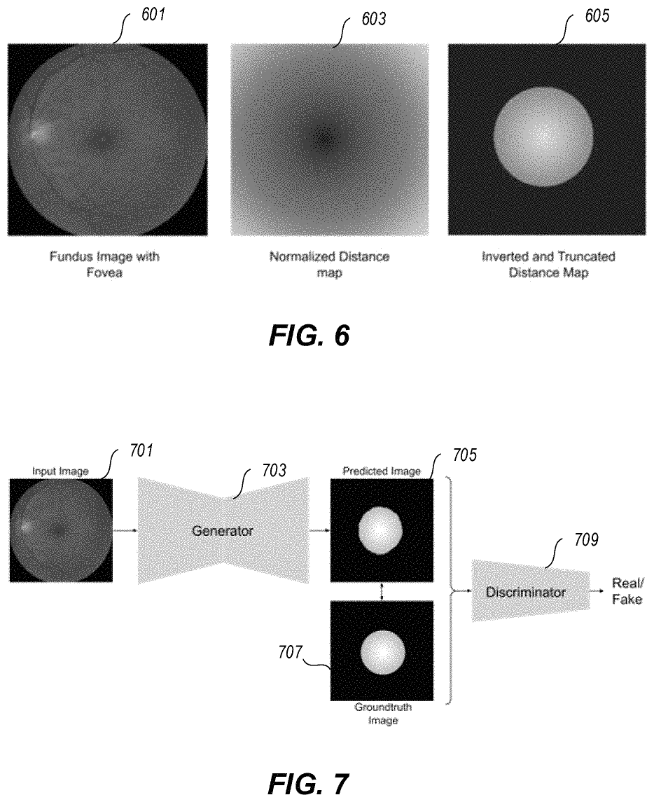

FIG. 6 is an image diagram showing a sample distance map used by the facility to locate the fovea in some embodiments. FIG. 6 includes a raw fundus image 601, a normalized distance map 603, and an inverted and truncated distance map 605. The facility uses the images obtained in act 501, such as fundus image 601, to create the distance maps. In some embodiments, the distance maps for each image are the same size as the image. In some embodiments, the facility uses ground truth point coordinates to generate the distance maps.

In some embodiments, the facility normalizes the distance map so that the distance map, to generate the normalized distance map 603. In some embodiments, when training classification networks used for the macula extraction module, the facility inverts the normalized distance map to improve training by making distances from points nearer to the fovea have higher values. In some embodiments, when training classification networks used for the macula extraction module, the facility truncates the distance map to improve training by forcing the distance map to contain only a predetermined radius around the fovea, such as the inverted and truncated distance map 605.

In some embodiments, at act 503, the facility uses image-to-image translation to locate the fovea. In some embodiments, the image-to-image translation is paired image to image translation, such as the method used in Shankaranarayana et al. Shankaranarayana, S. M., Ram, K., Mitra, K., Sivaprakasam, M.: Joint optic disc and cup segmentation using fully convolutional and adversarial networks. In: Fetal, Infant and Ophthalmic Medical Image Analysis, pp. 168-176. Springer (2017), herein incorporated by reference in its entirety. In some embodiments, the facility uses a GAN framework to along with image-to-image translation to locate the fovea.

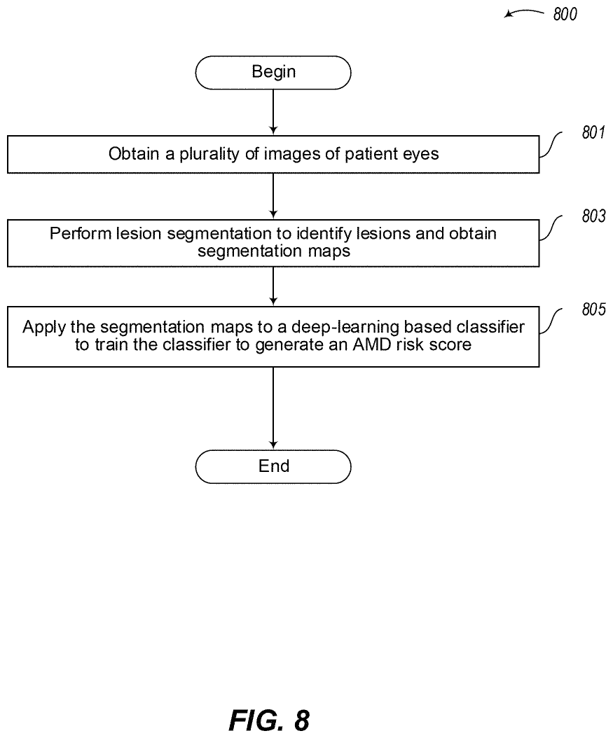

FIG. 7 is a display diagram depicting a GAN framework used to locate the fovea, used by the facility in some embodiments. The GAN framework includes an input image 701, a generator 703, a predicted image 705, a ground truth image 707, and a discriminator 709. The GAN framework is used to synthesize data used for image-to-image translation and locate the fovea of a subject patient's eye. The input image 701 is one of the images obtained in act 501. The generator 703 generates different images based on the input image, such as the predicted image 705. The predicted image 705 is compared to a ground truth image 707 by the discriminator 709, which determines whether the image is real or fake. This determination is used to assist in training a classification model to locate the fovea and generate an AMD risk score.

Returning to FIG. 5, at act 505, the facility crops the images around the fovea. In some embodiments, the facility applies a Euclidean distance transform computed from the fovea location to crop the images. In such embodiments, by cropping the images, the facility is able to train the deep learning classifier as a fine-grained classifier which focuses on the macular region.

At act 507, the facility applies the cropped images to a deep-learning based classifier to train the classifier to generate a risk score for AMD. In some embodiments, the deep-learning based classifier is a fine-grained classifier trained with cropped images focusing on the macular region. After act 507, the process ends.

FIG. 8 is a flow diagram showing a process performed by the facility in some embodiments to create a lesion extraction module used to generate a third AMD risk score. At act 801, the facility obtains a plurality of images of patient eyes. The facility performs act 801 in a similar manner to acts 301 and 501.

At act 803, the facility performs lesion segmentation to identify lesions in the eyes depicted in the plurality of images and obtain segmentation maps for the images. In some embodiments, the facility uses a GAN to create the segmentation maps.

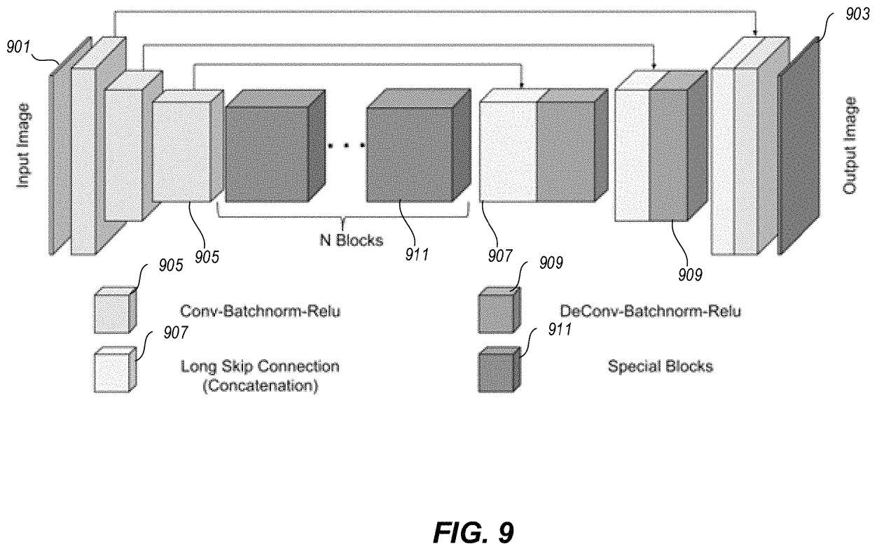

FIG. 9 is a lesion segmentation GAN framework used by the facility in some embodiments. In some embodiments, the facility uses the GAN framework depicted in FIG. 9 to locate the fovea instead of the framework depicted in FIG. 7. The lesion segmentation GAN framework includes an input image 901, an output image 903, convolution layers 905, concatenation layers 907, deconvolution layers 909, and special blocks 911. The input image 901 is one of the images obtained in act 801. The output image 903 is a segmentation map which identifies lesions present in the input image 901.

The convolution layers 905 are used as coarse feature extraction layers. In some embodiments, the GAN includes multiple convolution layers 905, such as the three layers present in FIG. 9. In some embodiments, the facility uses strided convolution for downsampling in later convolution layers 905. For example, in the GAN depicted in FIG. 9, the facility uses strided convolution on the second and third layers.

The special blocks 911 are used in both the encoding path for downsampling and the decoding path for upsampling. Each special block consists of two convolutional blocks and one skip connection with 3×3 filters and a stride of 1 followed by batch normalization and a Relu activation function. These special blocks make improvements to the existing u-net architecture by replacing the normal convolutional blocks with residual blocks. The special blocks may be similar to the special blocks used in He, K. et al. and Shankaranarayana, S. M., et al., previously incorporated by reference. He, K., Zhang, X., Ren, S., Sun, J.: Deep residual learning for image recognition. In: Proceedings of the IEEE conference on computer vision and pattern recognition. pp. 770-778 (2016), herein incorporated by reference in its entirety.

In some embodiments, in the encoding path, while downsampling, the facility uses a 4x4 convolution with a stride of 2 followed by a batch normalization Relu operation and doubles the number of filters in each layer after downsampling. In some embodiments, the facility doubles the number of filters only until it reaches a predetermined number of filters, such as, for example, 512. In such an example, all subsequent layers have only 512 filters. Thus, the facility is able to keep the number of parameters low while still maintaining accuracy normally achieved by using more parameters.

In some embodiments, in the decoding path, while upsampling the facility reverses the encoding path, and each layer is matched 1 to 1. In some embodiments, the facility uses different dropout rates in the initial layers of the decoding path.

The deconvolutional layers 909 are used to upsample the images after processing them with the special blocks 911. In some embodiments, the facility additionally uses long skip connections to recover information lost during downsampling. In some embodiments, in the decoder, the facility uses deconvolutional filters with upsampling on the feature maps to predict the final segmented image. In some embodiments, the facility uses a 1×1 convolution followed by a tanh activation in the last layer of the decoders to obtain the segmentation

In some embodiments, at least a portion of the layers, such as, for example, all of the layers except the final layer, are followed by batch normalization, Relu operations, or both. In some embodiments, the final layer is followed by tanh activation. In some embodiments, the facility trains the GAN separately for each type of lesion, and provides segmentation maps for each type of lesion separately. In some embodiments, the facility discards segmentation predictions within the segmentation map where the lesion area is less than a predetermined value.

Returning to FIG. 8, at act 805, the facility applies the segmentation maps and extracted lesions to train the deep-learning based classifier to generate a risk score for AMD. After act 805, the process ends.

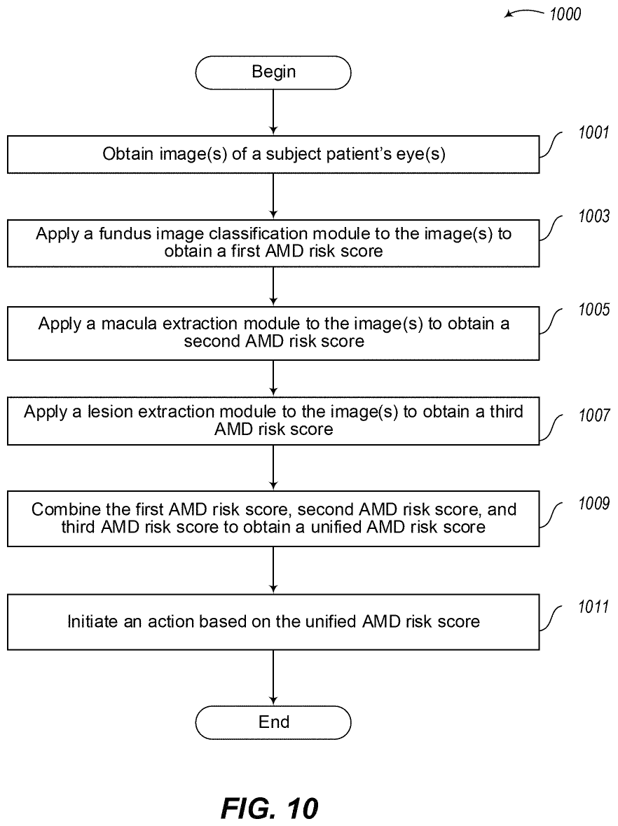

FIG. 10 is a flow diagram showing a process performed by the facility in some embodiments to use an ensemble model to obtain an AMD risk score for a subject patient. At act 1001, the facility obtains one or more images of at least one of a subject patient's eyes. In some embodiments, the facility obtains the images by using color fundus imaging, OCT, or other imaging techniques for obtaining images of eyes.

At act 1003, the facility applies a fundus image classification module to at least a portion of the one or more images to obtain a first AMD risk score. In some embodiments, the facility augments at least a portion of the one or more images in a similar manner to act 303 before applying the images to the fundus image classification module.

At act 1005, the facility applies a macula extraction module to at least a portion of the one or more images to obtain a second AMD risk score. In some embodiments, the facility applies the entire image to the macula extraction module.

In some embodiments, as part of applying the macula extraction module to the images, the facility locates the fovea in each image in a similar manner to act 503. In some embodiments, as part of applying the macula extraction module to the images, the facility crops the image around the fovea in a similar manner to act 505.

At act 1007, the facility applies a lesion segmentation module to at least a portion of the one or more images to obtain a third AMD risk score. In some embodiments, as part of applying the lesion segmentation module to the images, the facility generates segmentation maps of the images, which are used by the lesion extraction module to generate the third AMD risk score.

In some embodiments, the portions of the one or more images used in each of acts 1003, 1005, and 1007, contain at least one image in common. In some embodiments, the portions the one or more images used in each of acts 1003, 1005, and 1007, do not contain any images in common. In some embodiments, acts 1003, 1005, and 1007 are performed in parallel. In some embodiments, acts 1003, 1005, and 1007 are performed sequentially.

At act 1009, the first AMD risk score, second AMD risk score, and third AMD risk score are combined to obtain a unified AMD risk score. In some embodiments, the facility uses an average, such as a weighted average, mean, median, etc. to combine the AMD risk scores.

At act 1011, the facility initiates an action based on the unified AMD risk score. In some embodiments, the action includes presenting the risk score to a medical practitioner, a patient, etc. In some embodiments, the action includes transmitting the risk score to a medical device or system, and the risk score is used to change or alter the diagnosis, treatment, or medical advice provided to a patient. After act 1011, the process ends.

In some embodiments, in addition to providing the AMD risk scores, the facility also presents the segmented retinal lesions to a medical provider. In some embodiments, the facility uses a system which employs OCT for the automated detection of AMD, such as the method proposed in Wang, W., et al., to combine OCT and fundus imaging modalities for the detection of AMD. Wang, W., Xu, Z., Yu, W., Zhao, J., Yang, J., He, F., Yang, Z., Chen, D., Ding, D., Chen, Y., et al.: Two-stream cnn with loose pair training for multimodal and categorization.

In: International Conference on Medical Image Computing and Computer-Assisted Intervention. pp. 156-164. Springer (2019), herein incorporated by reference in its entirety.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system for diagnosing AMD in a subject patient, the system comprising:
a memory storing one or more patient images for the subject patient, the one or more patient images depicting at least one of the subject patient's eyes;
at least one processor configured to:
apply an image-based classifier to at least one patient image of the one or more patient images to obtain a first AMD risk score;
identify a macular region based on at least one patient image of the one or more patient images;
apply a deep learning-based classifier to the identified macular region to obtain a second AMD risk score;
identify lesions based on at least one patient image of the one or more patient images;
apply a deep learning-based classifier to the identified lesions to obtain a third AMD risk score; and
combine the first AMD risk score, second AMD risk score, and third AMD risk score to obtain a unified AMD risk score.

2. The system of claim 1, further comprising:
applying a fundus image classification module which includes the image-based classifier to obtain the first AMD risk score.

3. The system of claim 2, wherein applying the fundus image classification module further comprises:
altering the at least one patient image of the one or more patient images prior to applying the image-based classifier to the at least one patient image to obtain the first AMD risk score.

4. The system of claim 1, further comprising:
applying a macula extraction module to at least one patient image of the one or more patient images to identify the macular region, wherein the macula extraction module includes the deep learning-based classifier used to obtain the second AMD risk score.

5. The system of claim 4, further comprising:
applying a Generative Adversarial Network (GAN) included in the macula extraction module to identify the macular region.

6. The system of claim 4, wherein applying the macula extraction module further comprises:
generating a distance map based on at least one patient image of the one or more patient images; and
using the generated distance map to locate at least one fovea of at least one of the subject patient's eyes.

7. The system of claim 4, wherein applying the macula extraction module further comprises:
employing image-to-image translation to locate at least one fovea of at least one of the subject patient's eyes.

8. The system of claim 1, further comprising:
applying a lesion extraction module to at least one patient image of the one or more patient images to identify the lesions, wherein the lesion extraction module includes the deep learning-based classifier used to obtain the third AMD risk score.

9. The system of claim 8, further comprising:
applying a Generative Adversarial Network (GAN) included in the lesion extraction module to identify the lesions.

10. The system of claim 8, further comprising:
applying a fully convolutional network included in the lesion extraction module to identify the lesions.

11. The system of claim 1, wherein at least one patient image of the one or more patient images is a color fundus image.

12. The system of claim 1, wherein at least one patient image of the one or more patient images is obtained by using optical coherence tomography (OCT).

13. One or more instances of computer-readable media collectively having contents configured to cause a computing device to perform a method for creating modules used to diagnose AMD, the method comprising:
obtaining one or more patient images for a subject patient, the one or more patient images depicting at least one of the subject patient's eyes;
generating a fundus classification module to obtain a first AMD risk score, wherein the fundus classification module is configured to:
apply an image-based classifier to at least one patient image of the one or more patient images to obtain the first AMD risk score;
generating a macula extraction module to obtain a second AMD risk score, wherein the macula extraction module is configured to:
identify a macular region based on at least one patient image of the one or more patient images; and
apply a deep learning-based classifier to the identified macular region to obtain the second AMD risk score;
generating a lesion extraction module to obtain a third AMD risk score, wherein, the lesion extraction module is configured to:
identify lesions based on at least one patient image of the one or more patient images;
apply a deep learning-based classifier to the identified lesions to obtain the third AMD risk score;
applying the one or more patient images to the fundus classification module to obtain the first AMD risk score;
applying the one or more patient images to the macula extraction module to obtain the second AMD risk score;
applying the one or more patient images to the lesion extraction module to obtain the third AMD risk score; and
combining the first AMD risk score, second AMD risk score, and third AMD risk score to obtain a unified AMD risk score.

14. The one or more instances of computer-readable media of claim 13, wherein the fundus classification module is further configured to:
alter at least one patient image of the one or more patient images.

15. The one or more instances of computer-readable media of claim 13, wherein the macula extraction module is further configured to:
use a GAN to identify the macular region.

16. The one or more instances of computer-readable media of claim 13, wherein the macula extraction module is further configured to:
generate a distance map based on at least one patient image of the one or more patient images; and
use the generated distance map to locate at least one fovea of at least one of the subject patient's eyes.

17. The one or more instances of computer-readable media of claim 13, wherein the macula extraction module is further configured to:
employ image-to-image translation to locate at least one fovea of at least one of the subject patient's eyes.

18. The one or more instances of computer-readable media of claim 13, wherein the lesion extraction module is further configured to:
apply a GAN included in the lesion extraction module to identify the lesions.

19. The one or more instances of computer-readable media of claim 13, wherein the lesion extraction module is further configured to:
apply a fully convolutional network included in the lesion extraction module to identify the lesions.

20. The one or more instances of computer-readable media of claim 13, wherein at least one image of the obtained one or more images is a color fundus image.

21. The one or more instances of computer-readable media of claim 13, wherein at least one image of the obtained one or more images is obtained by using OCT.

22. One or more storage devices collectively storing an AMD diagnosis data structure, the data structure comprising:
information representing one or more patient images for a subject patient, the one or more patient images depicting at least one eye of the subject patient's eyes;
information representing a first AMD risk score, the first AMD risk score being obtained by a fundus image classification module, wherein the fundus image classification module obtains the first AMD risk score by applying an image-based classifier to at least one patient image of the one or more patient images;
information representing a second AMD risk score, the second AMD risk score being obtained by a macula extraction module configured to:
identify a macular region based on at least one patient image of the one or more patient images; and
apply a deep learning-based classifier to the identified macular region to obtain a second AMD risk score; and
information representing a third AMD risk score, the third AMD risk score being obtained by a lesion extraction module configured to:
identify lesions based on at least one patient image of the one or more patient images; and
apply a deep learning-based classifier to the identified lesions to obtain a third AMD risk score,
such that the information representing the first AMD risk score, second AMD risk score, and third AMD risk score are able to be combined to obtain a unified AMD risk score.

23. The one or more storage devices of claim 22, wherein at least one patient image of the one or more patient images is a color fundus image.

24. The one or more storage devices of claim 22, wherein at least one patient image of the one or more patient images is obtained by using OCT.

25. The one or more storage devices of claim 22, wherein the AMD diagnosis data structure further comprises:
information representing a GAN, such that the macula extraction module uses the GAN to identify the macular region.

26. The one or more storage devices of claim 22, wherein the AMD diagnosis data structure further comprises:
information representing a distance map, such that the macula extraction module uses the distance map to identify a fovea of at least one of the subject patient's eyes.

27. The one or more storage devices of claim 22, wherein the AMD diagnosis data structure further comprises:
information representing a GAN, such that the lesion extraction module uses the GAN to identify lesions.
